(19) United States
(12) Patent Application Publication
(10) Pub. No.: US 2025/0166179 A1
(43) Pub. Date: May 22, 2025

(54) METHOD FOR ASSISTING DETECTION OF PERIODONTAL DISEASES

(71) Applicant: Chung Shan Medical University, Taichung City (TW)

(72) Inventors: Chia-Tze KAO, Taichung City (TW); Chih-Jen TSENG, Taichung City (TW)

(73) Assignee: Chung Shan Medical University, Taichung City (TW)

(21) Appl. No.: 18/944,072

(22) Filed: Nov. 12, 2024

(30) Foreign Application Priority Data
Nov. 16, 2023 (TW) 112144288

(51) Int. Cl. G06T 7/00; A61B 5/00

(52) U.S. Cl. CPC G06T 7/0012; A61B 5/4547; G06T 2207/20081; G06T 2207/20084; G06T 2207/30036

(57) ABSTRACT

A method for assisting detection of periodontal diseases includes: with respect to a to-be-processed image that consists of a teeth section and an alveolar bone section, generating a first reference curve and a second reference curve based on grayscale gradient values associated with pixels of the to-be-processed image; generating an alveolar boundary curve; obtaining a main initial value and a main shrinkage value based on the first reference curve, the second reference curve and the alveolar boundary curve, and calculating a main assessment as a ratio of the main shrinkage value to the main initial value.

### CROSS-REFERENCE TO RELATED APPLICATION

This application claims priority to Taiwanese Invention Patent Application No. 112144288, filed on Nov. 16, 2023, the entire disclosure of which is incorporated by reference herein.

### FIELD

The disclosure relates to a method for assisting dentistry, and more particularly to a method for assisting detection of periodontal diseases.

### BACKGROUND

In the field of dentistry, the application of detecting periodontal diseases for a patient is typically done by a dentist by first using an odontoscope to inspect the gum in the oral cavity of the patient to determine whether swelling or inflammation is present. Additionally, an X-ray dental image (known as a panoramic radiograph or panoramic X-ray) of the patient may be taken and inspected by the dentist to determine whether syndromes such as periodontal bone loss and apical foramen inflammation are present. The dentist may also operate a periodontal probe to determine a pocket depth of any periodontal pocket, so as to evaluate the severity of the periodontal diseases.

It is noted that the above procedures typically involve subjective judgement of the dentist and the feelings of the patient, and therefore the diagnosis may not be accurate.

### SUMMARY

In order to eliminate potential medical disputes resulting from the subjective nature of such kind of diagnosis, a more objective method for assisting diagnosis may be desirable

Therefore, an object of the disclosure is to provide a method for assisting detection of periodontal diseases that is done more objectively.

According to one embodiment of the disclosure, the method for assisting detection of periodontal diseases is implementing using a processor of a computer system and comprising steps of:
a) obtaining a to-be-processed image that includes a teeth section that is associated with one row of teeth of a patient, and an alveolar bone section that is adjacent to the teeth section, wherein the teeth section is partitioned into a plurality of embedded areas and a plurality of exposed areas, each of the embedded areas is defined to cover a part of a corresponding tooth that is within a corresponding alveolar bone, and overlaps a part of the alveolar bone section, and each of the exposed areas extends from a corresponding one of the embedded areas, and is defined to cover a part of the corresponding tooth that extends from the corresponding alveolar bone;
b) generating a first reference curve, and a second reference curve, wherein
the first reference curve is generated by detecting, for each of the embedded areas included in the teeth section, a tip point, and connecting the tip points of the embedded areas to obtain the first reference curve, and
the second reference curve is generated by, determining, for each of the exposed areas and along a first direction, at least one boundary pixel at which a grayscale gradient value with an adjacent pixel is the largest among pixels in the exposed area and which is located near a junction between the exposed area and the corresponding one of the embedded areas, and connecting the boundary pixels of the exposed areas to obtain the second reference curve;
c) generating an alveolar boundary curve by detecting, for each of the exposed areas of the teeth section, at least one border pixel at which a grayscale gradient value with an adjacent pixel is the largest among the pixels in the exposed area and which is located near a contour of the alveolar bone section, thereby resulting in a plurality of border pixels, and connecting the border pixels of the exposed areas to obtain the alveolar boundary curve, wherein the border pixels of the exposed areas of the teeth section are located between the first reference curve and the second reference curve; and
d) obtaining a main initial value and a main shrinkage value based on the first reference curve, the second reference curve and the alveolar boundary curve, and calculating a main assessment as a ratio of the main shrinkage value to the main initial value, wherein the main initial value is an average distance between the first reference curve and the second reference curve, the main shrinkage value is a largest distance between the first reference curve and the alveolar boundary curve.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a flow chart illustrating steps of an exemplary method for assisting detection of periodontal diseases according to one embodiment of the disclosure.

FIG. 2 illustrates an exemplary to-be-processed image according to one embodiment of the disclosure.

FIG. 3 illustrates an exemplary labeled image according to one embodiment of the disclosure.

FIG. 4 illustrates operations for generating a first reference curve on the to-be-processed image.

FIG. 5 illustrates operations for generating a second reference curve on the to-be-processed image.

FIG. 6 illustrates operations for generating an auxiliary curve on the to-be-processed image.

FIG. 7 illustrates operations for generating an alveolar boundary curve on the to-be-processed image.

FIG. 8 illustrates operations for obtaining a main initial value and a main shrinkage value based on the first reference curve, the second reference curve and the alveolar boundary curve, and calculating a main assessment.

FIG. 9 illustrates operations for obtaining a plurality of sub-initial values and a plurality of sub-shrinkage values based on the first reference curve, the second reference curve and the alveolar boundary curve, and calculating a plurality of sub-assessments.

FIG. 10 is a block diagram of a computer system for use to implement a method for assisting detection of periodontal diseases according to one embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “coupled to” or “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

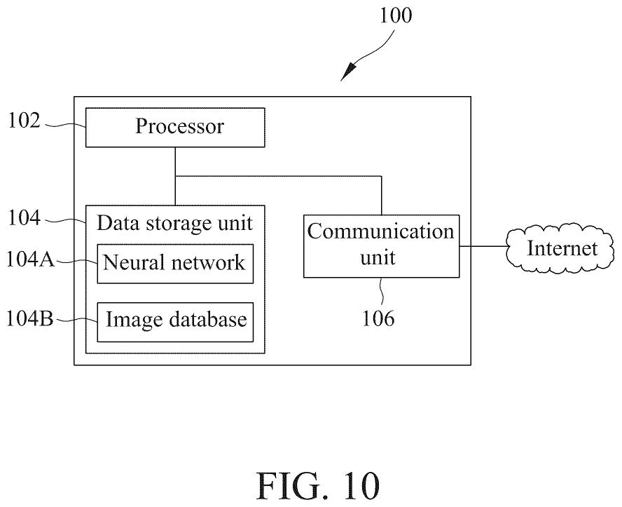

FIG. 10 is a block diagram of a computer system 100 used to implement a method for assisting detection of periodontal diseases according to one embodiment of the disclosure. In this embodiment, the computer system 100 may be embodied using a server, a personal computer, a laptop or other suitable equipment.

In the embodiment of FIG. 10, the computer system 100 includes a processor 102, a data storage unit 104, and a communication unit 106.

The processor 102 may be embodied using a central processing unit (CPU), a microprocessor, a microcontroller, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc.

The data storage unit 104 is connected to the processor 102, and may be embodied using, for example, random access memory (RAM), read only memory (ROM), programmable ROM (PROM), firmware, flash memory, etc. In this embodiment, the data storage unit 104 stores a software application, a neural network 104A and an image database 104B therein. The software application includes instructions that, when executed by the processor 102, cause the processor 102 to implement the operations as described below.

The neural network 104A may be prepared using TensorFlow as a deep learning framework, compiled using a supported programming language such as Python.

The image database 104B includes a plurality of images used for training the neural network 104A and/or for subsequent processing. In this embodiment, each of the images may be extracted from a panoramic radiography (also known as a panoramic X-ray) dental image, may be represented in the form of a grayscale image, and may be categorized into one of a to-be-processed image and a labeled image.

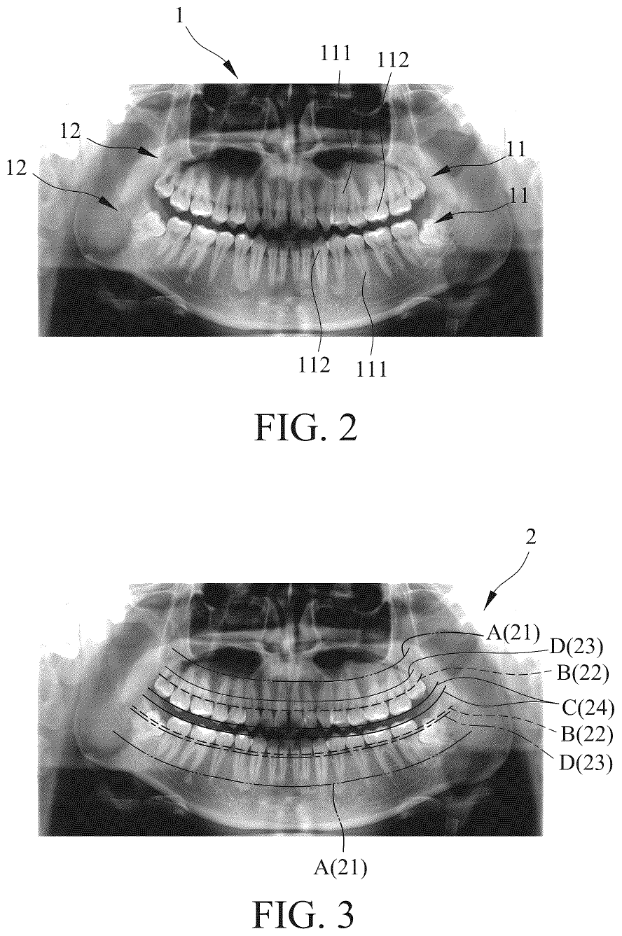

FIG. 2 illustrates an exemplary to-be-processed image 1 according to one embodiment of the disclosure. FIG. 3 illustrates an exemplary labeled image 2 according to one embodiment of the disclosure. It is noted that in other embodiments, the images may be other images that clearly illustrate the teeth and the alveolar bone of the patient. In the embodiment of FIG. 10, the image database 104B includes at least one to-be-processed image 1 and a plurality of labeled images 2, but it is not limited to such.

In the embodiment of FIG. 2, the to-be-processed image 1 includes a plurality of pixels each being represented by a grayscale value, and the to-be-processed image 1 may include a number of different sections. Additionally, for the propose of illustration, the to-be-processed image 1 as shown in the drawings may not be in the precise grayscale values, but in use, the to-be-processed image 1 is indeed a grayscale image.

Specifically, the to-be-processed image 1 includes two teeth sections 11 that are associated with the upper row of teeth and the lower row of teeth of a patient, respectively, and two alveolar bone sections 12 that are adjacent to the two teeth sections 11, respectively. The teeth sections 11 are defined to cover the upper teeth and the lower teeth, including the crowns and the roots. The alveolar bone sections 12 are defined to the portion of bone containing the sockets on the jaw bones, including maxilla and mandible.

Each of the teeth sections 11 may be further partitioned into a number of embedded areas 111 and a number of exposed areas 112. Each of the embedded areas 111 is defined to cover a part of a corresponding tooth that is within the corresponding alveolar bone (e.g., a root of the tooth), and overlaps a part of a corresponding one of the alveolar bone sections 12. Each of the exposed areas 112 extends from a corresponding one of the embedded areas 111, and is defined to cover a part of the corresponding tooth that extends from the corresponding alveolar bone (e.g., a crown of the tooth).

In the embodiment of FIG. 3, the labeled image 2 is also a grayscale panoramic radiography image, and includes a number of label lines. The label lines are associated with different dental parts on the labeled image 2. Specifically, as shown in FIG. 3, the labeled lines on the labeled image 2 include two first curves 21, two second curves 22, two third curves 23, and two auxiliary curves 24. The labeled lines may be categorized into two sets that respectively correspond to the upper and lower rows of teeth, and each set includes one first curve 21, one second curve 22, one third curve 23 and one auxiliary curve 24. In some embodiments, the labeled lines may be manually drawn by a person or automatically drawn using a computer device (e.g., the computer system 100) executing a software application such as an image recognition software.

Each of the first curves 21 indicates the apical foramina of the corresponding row of teeth. Each of the second curves 22 indicates an edge of the interdental gingiva associated with the corresponding row of teeth (i.e., the edge of the interdental gingiva separates the teeth and the gums). Each of the third curves 23 indicates a contour of an alveolar bone associated with the corresponding row of teeth. Each of the auxiliary curves 24 indicates the cusps of the corresponding row of teeth.

The communication unit 106 is connected to the processor 102, and may include one or more of a radio-frequency integrated circuit (RFIC), a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G), the fourth generation (4G) or fifth generation (5G) of wireless mobile telecommunications technology, or the like. In one embodiment, the communication unit 106 enables the computer system 100 to establish a communication with other electronic devices via a network (e.g., the Internet).

In use, when it is desired to detect periodontal diseases for a patient, a dentist may first obtain an image of the patient (which may serve as the to-be-processed image 1), and implement a method for assisting detection of periodontal diseases using the to-be-processed image 1, in order to more objectively determine whether the patient indeed has periodontal diseases. For example, some of the periodontal diseases may result in syndromes that can be detected on the to-be-processed image 1, such as alveolar bone loss (which may be indicated by a shape and a size of one of the alveolar bone sections 12 included in the to-be-processed image 1).

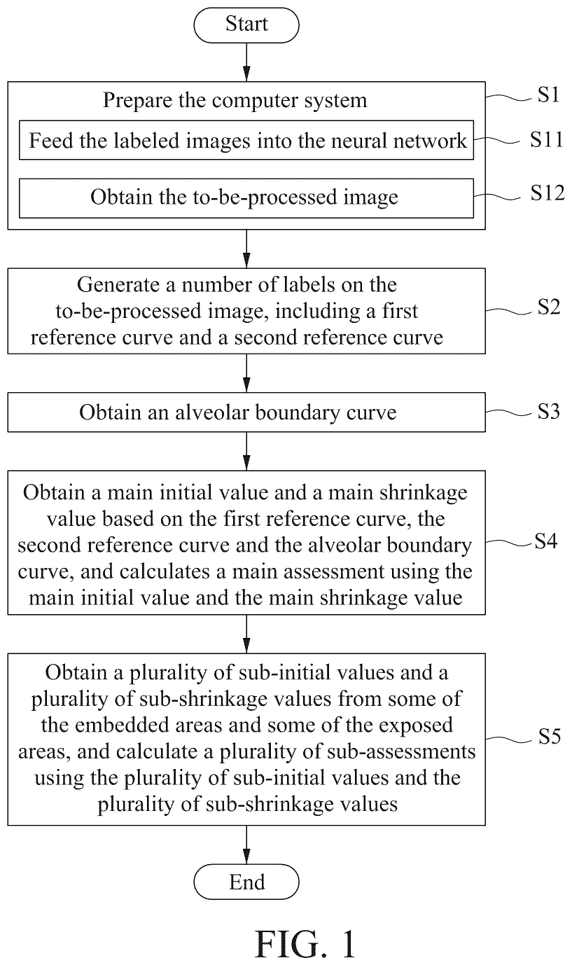

FIG. 1 is a flow chart illustrating steps of an exemplary method for assisting detection of periodontal diseases according to one embodiment of the disclosure. In some embodiments, the method is to be implemented using the computer system 100 of FIG. 10. In some embodiments, prior to the implementation of the method, the image database 104B includes a plurality of labeled images 2.

According to some embodiments, the computer system 100 is not yet prepared, and therefore in step S1, the computer system 100 is prepared. Specifically, the operations of step S1 includes a sub-step S11 and a sub-step S12. In sub-step S11, the labeled images 2 are fed into the neural network 104A for training the same. In this embodiment, the training of the neural network 104A may be done in a manner of deep learning, but is not limited to such. Afterward, the trained neural network 104A may be used for the operations below.

Then, in sub-step S12, the processor 102 obtains the to-be-processed image 1. Specifically, in a case where the data storage unit 104 stores a data file that is a dental imaging file of the patient obtained using panoramic radiography, computer topography (CT) or another imaging technique and that is in the format of Digital Imaging and Communications in Medicine (DICOMO), the processor 102 executes an image extraction operation on the data file, so as to obtain an extracted image.

Then, the processor 102 implements an image processing operation on the extracted image. In this embodiment, the image processing operation may include conducting normalization with respect to the grayscale values of the pixels of the extracted image in a hue, saturation, value (HSV) space. As such, the extracted image extracted from the data file may be converted into a grayscale image (i.e., the to-be-processed image 1), with contours of the two teeth sections 11 (including the contours of the exposed areas 112 and the corresponding the embedded areas 111) and the contours of the two alveolar bone sections 12 defined or identified. Also, a grayscale gradient value of two adjacent pixels (i.e., a difference between the grayscale values of the two adjacent pixels) in each of the two teeth sections 11 may be calculated. In this manner, the parts of the to-be-processed image 1 with higher grayscale values (i.e., the parts of the extracted image with greater brightness) may be kept after the image processing operation is completed. It is noted that in cases where the to-be-processed image 1 is already stored in the data storage unit 104, the image extraction operation and/or the image processing operation may be omitted.

It is noted that in some embodiments, the computer system 100 may already be prepared, and therefore the operations of step S1 may be omitted.

Then, in step S2, the processor 102 generates a number of labels on the to-be-processed image 1, with respect to each of the teeth sections 11 and the corresponding one of the alveolar bone sections 12. For the sake of brevity, the following operations will be described with respect to one of the teeth sections 11 and the corresponding one of the alveolar bone sections 12, but in reality, the processor 102 may implement the operations on both of the teeth sections 11 and both of the alveolar bone sections 12.

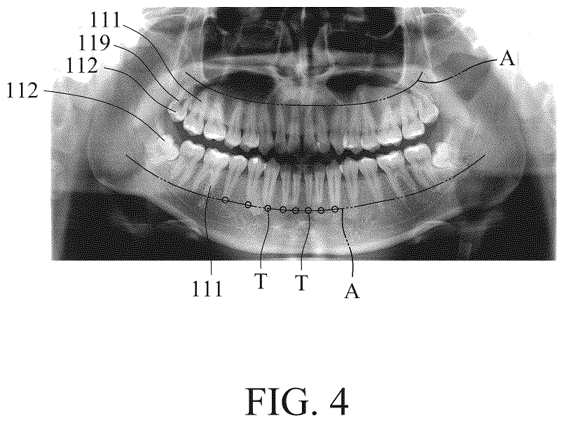

As shown in FIG. 4, in step S2, the processor 102 first executes an edge detection operation on the teeth section 11 to define a first reference curve A (for the entire to-be-processed image 1, a total of two first reference curves A would be defined). Specifically, the processor 102 may detect, for each of the embedded areas 111 included in the teeth section 11, a tip point T (that indicates an apical foramen of the corresponding tooth), and then the processor 102 may connect the tip points T to obtain the first reference curve A.

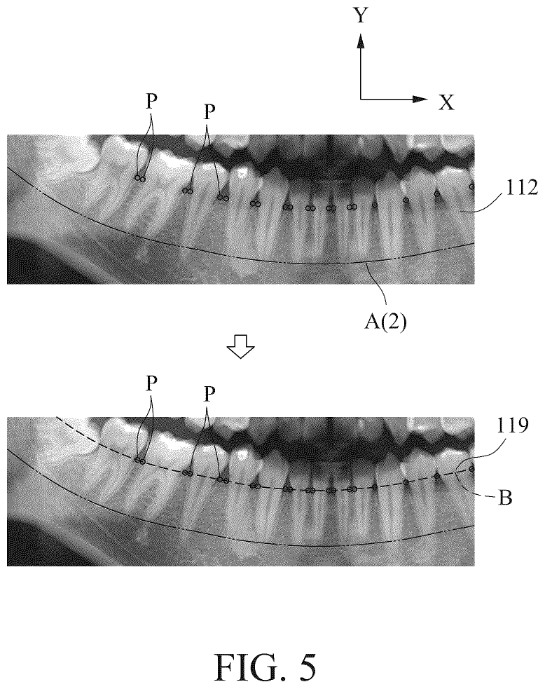

Next, as shown in FIG. 5, the processor 102 determines, for each of the exposed areas 112 and along a first direction (e.g., the X direction indicated on FIG. 5), at least one boundary pixel P at which a grayscale gradient value with an adjacent pixel is the largest among pixels in the exposed area 112 and which is located near a junction between the exposed area 112 and an adjacent one of the embedded areas 111, resulting in a plurality of boundary pixels P. In use, for each of the exposed areas 112, the processor 102 may obtain a plurality of grayscale gradient values for the pixels in the exposed area 112 along the first direction, and then determine the at least one boundary pixel P by finding one or more pixels with the largest grayscale gradient value. Specifically, since a root of a tooth and the adjacent interdental gingiva have starkly different grayscale values on the to-be-processed image 1, by identifying the boundary pixel P, a positional relationship between each of the exposed areas 112 and the interdental gingiva may be obtained (i.e., the boundary pixel P indicates a junction between a tooth and the nearby interdental gingiva). It is noted that in some embodiments, due to the different alignments of the teeth, some of the boundary pixels P may be very close to each other. Then, the processor 102 obtains a second reference curve B on the teeth section 11 by connecting the boundary pixels P of the exposed areas 112.

In some alternative embodiments, the processor 102 further, for each of the exposed areas 112, defines a cervical margin 119 that separates the crown and the root of the tooth (that is to say, a plurality of cervical margins 119 are obtained). It is noted that the cervical margins 119, by definition, also separate the embedded areas 111 and the corresponding exposed areas 112. Additionally, the detection of the cervical margins 119 using existing image detection is well known in the related art, details thereof are omitted herein for the sake of brevity.

Then, the processor 102 obtains the second reference curve B on the teeth section 11 by connecting the boundary pixels P of the cervical margins 119. Generally, the second reference curve B indicates the boundary of interdental gingiva associated with the corresponding row of teeth of the patient.

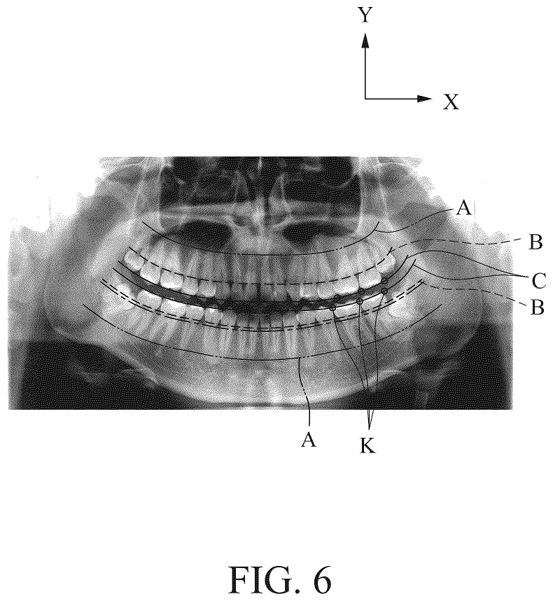

In some embodiments, the processor 102 further obtains an auxiliary curve C on the teeth section 11 by detecting, for each of the exposed areas 112 along a second direction (e.g., the Y direction indicated on FIG. 6), at least one edge pixel K at which a grayscale gradient value with an adjacent pixel is the largest among the pixels in the exposed area 112 and which is located near the contour of the exposed area 112, resulting in a plurality of edge pixels K. Then, the processor 102 connects the edge pixels K to obtain the auxiliary curve C. Generally, the auxiliary curve C indicates the cusps of the corresponding row of teeth of the patient.

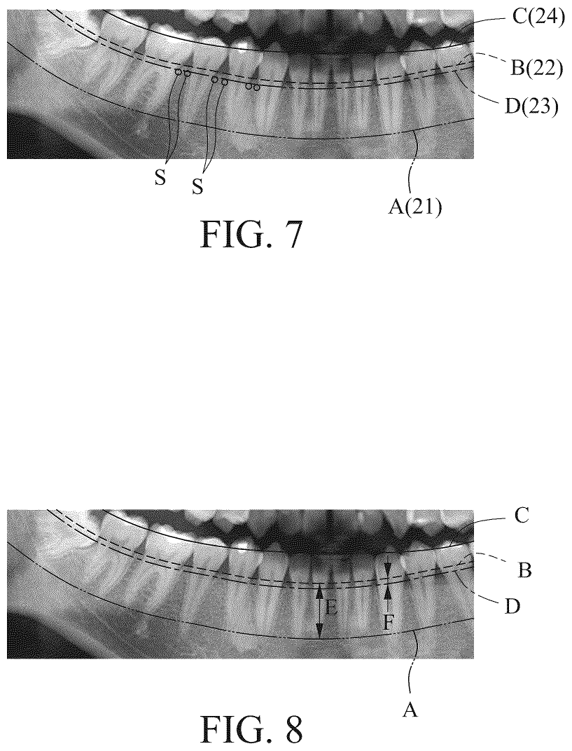

Then, in step S3, the processor 102 obtains an alveolar boundary curve D. In some embodiments, step S3 includes detecting, for each of the exposed areas 112 (e.g., the Y direction indicated on FIG. 7), at least one border pixel S at which a grayscale gradient value with an adjacent pixel is the largest among the pixels in the exposed area 112 and which is located near the contour of a corresponding one of alveolar bone sections 12, resulting in a plurality of border pixels S. It is noted that each of the border pixels S is located between the first reference curve A and the second reference curve B. Then, the processor 102 connects the border pixels S to obtain the alveolar boundary curve D. Generally, the alveolar boundary curve D indicates boundary of the alveolar bone associated with the corresponding row of teeth of the patient.

In reality, after the above steps are implemented, as shown in FIG. 3, the to-be-processed image 1 would have two first reference curves A which correspond respectively with the two first curves 21, two second reference curves B which correspond respectively with the two second curves 22, two auxiliary curves C which correspond respectively with the two auxiliary curves 24, and two alveolar boundary curves D which correspond respectively with the two third curves 23. That is to say, after using the plurality of labeled images 2 to train the neural network 104A, the above steps may be implemented by the processor 102 executing the neural network 104A to label corresponding curves on the to-be-processed image 1.

Then, in step S4, the processor 102 obtains a main initial value E and a main shrinkage value F based on the first reference curve A, the second reference curve B and the alveolar boundary curve D, and calculates a main assessment using the main initial value E and the main shrinkage value F. Specifically, as shown in FIG. 8, the main initial value E is defined as an average distance between the first reference curve A and the second reference curve B. The main shrinkage value F is defined as a largest distance between the second reference curve B and the alveolar boundary curve D. Then, the processor 102 calculates the main assessment using the formula:

$$Main Assessment = F / E \times 100 \%$$

Specifically, the largest distance between the second reference curve B and the alveolar boundary curve D indicates a largest depth by which the alveolar bone has shrunk into the gingiva, and the ratio of the main shrinkage value F to the main initial value E indicates a percentage of shrinkage of the alveolar bone. Since one typical symptom of periodontal diseases is the shrinkage of the alveolar bone, using the computer system 100 to automatically process and examine the dental image of the patient may result in a quick determination of whether the patient might have periodontal diseases.

In some embodiments, the main assessment, which is indicated by a percentage, may be interpreted based on the following Table 1, but is not limited to such.

TABLE 1

| Main assessment | Diagnosis |
|---|---|
| <33% | Healthy or Light periodontal |
| | disease |
| 33%-66% | Mild periodontal disease |
| >66% | Severe periodontal disease |

In some embodiments, the method further includes the operations of step S5, in which the processor 102 obtains a plurality of sub-initial values G and a plurality of sub-shrinkage values H from some of the embedded areas 111 and some of the exposed areas 112, and calculates a plurality of sub-assessments using the plurality of sub-initial values G and the plurality of sub-shrinkage values H. It is noted that each of the sub-initial values G and a corresponding one of sub-shrinkage values H are obtained with respect to one embedded area 111 and the corresponding exposed area 112 (i.e., one tooth)

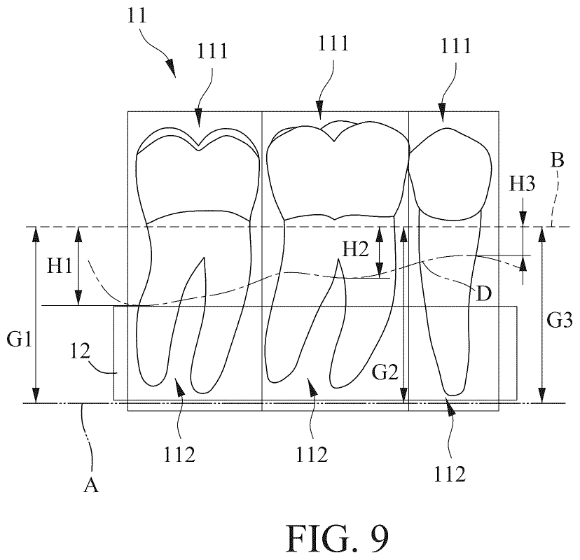

In one embodiment, as shown in FIG. 9, three exemplary teeth in a same row are illustrated. As such, the teeth section 11 includes three embedded areas 111 and three exposed areas 112. After the first reference curve A, the second reference curve B and the alveolar boundary curve D are labeled, the processor 102 obtains, with respect to each pair of the embedded area 111 and the corresponding exposed area 112 (i.e., each tooth), the sub-initial value G (which is an average distance between segments of the first reference curve A and the second reference curve B) that fall within the pair of the embedded area 111 and the corresponding exposed area 112 and the sub-shrinkage value H (which is a largest distance between the first reference curves A and the alveolar boundary curve D). In the embodiment of FIG. 9, three sub-initial values G (labeled as G1, G2 and G3) and three sub-shrinkage values H (labeled as H1, H2 and H3) are obtained.

Subsequently, a sub-assessment for each tooth may be calculated using the formula:

$$Sub - Assessment = G / H \times 100 \% .$$

Each sub-assessment indicates whether the symptom of periodontal diseases is found on the corresponding, specific tooth.

By calculating the main assessment and the sub-assessments, a dentist is able to determine both whether the alveolar bone in a row of teeth as a whole has shrunk, and the precise locations at which the alveolar bone has shrunk. This may be particularly useful for diagnosing periodontal diseases in earlier stages, during which symptoms (e.g., red, swollen gums, bleeding, bad breath, etc.) are less obvious to patients. In such a case, without proper examination, the periodontal diseases may become more severe, causing shrinkage of the alveolar bone. As such, the method as described in the embodiments may assist a dentist in quickly and objectively determining, using normalized data, whether an alveolar bone has shrunk to a degree that indicates the presence of periodontal diseases. As such, the dentist may be able to discover periodontal diseases earlier.

It is noted that in the embodiments, the to-be-processed image 1 is shown to have two teeth sections 11 and two alveolar bone sections 12; however, in other embodiments, an image with only one teeth section 11 and one alveolar bone section 12, corresponding to one row of teeth, can be used. In this case, the above method may yield one first reference curve A, one second reference curve B, one auxiliary curve C and one alveolar boundary curve D, and the subsequent calculation of the main assessment and the sub-assessments may be done with respect to the one row of teeth.

To sum up, embodiments of the disclosure provide a method for assisting detection of periodontal diseases. In the method, a computer system executing a neural network 104A is capable of labeling a to-be-processed image 1 (which may be or may originate from a dental image of a patient) with the first reference curve(s) A, the second reference curve(s) B, the auxiliary curve(s) C and the alveolar boundary curve(s) D, which are similar to the curves that are pre-labeled on the labeled images 2. Then, using the labeled curves, the computer system is capable of calculating a main assessment and, optionally, a plurality of sub-assessments, thereby determining whether or not, and how much and precisely where an alveolar bone has shrunk. As such, a dentist is able to quickly and objectively determine, using normalized data, whether an alveolar bone has shrunk to a degree that may indicates the presence of periodontal diseases.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for assisting detection of periodontal diseases, the method being implementing using a processor of a computer system and comprising steps of:
a) obtaining a to-be-processed image that includes a teeth section that is associated with one row of teeth of a patient, and an alveolar bone section that is adjacent to the teeth section, wherein the teeth section is partitioned into a plurality of embedded areas and a plurality of exposed areas, each of the embedded areas is defined to cover a part of a corresponding tooth that is within a corresponding alveolar bone, and overlaps a part of the alveolar bone section, and each of the exposed areas extends from a corresponding one of the embedded areas, and is defined to cover a part of the corresponding tooth that extends from the corresponding alveolar bone;
b) generating a first reference curve, and a second reference curve, wherein
the first reference curve is generated by detecting, for each of the embedded areas included in the teeth section, a tip point, and connecting the tip points of the embedded areas to obtain the first reference curve, and
the second reference curve is generated by, determining, for each of the exposed areas and along a first direction, at least one boundary pixel at which a grayscale gradient value with an adjacent pixel is the largest among pixels in the exposed area and which is located near a junction between the exposed area and the corresponding one of the embedded areas, and connecting the boundary pixels of the exposed areas to obtain the second reference curve;
c) generating an alveolar boundary curve by detecting, for each of the exposed areas of the teeth section, at least one border pixel at which a grayscale gradient value with an adjacent pixel is the largest among the pixels in the exposed area and which is located near a contour of the alveolar bone section, thereby resulting in a plurality of border pixels, and connecting the border pixels of the exposed areas to obtain the alveolar boundary curve, wherein the border pixels of the exposed areas of the teeth section are located between the first reference curve and the second reference curve; and
d) obtaining a main initial value and a main shrinkage value based on the first reference curve, the second reference curve and the alveolar boundary curve, and calculating a main assessment as a ratio of the main shrinkage value to the main initial value, wherein the main initial value is an average distance between the first reference curve and the second reference curve, the main shrinkage value is a largest distance between the first reference curve and the alveolar boundary curve.

2. The method as claimed in claim 1, further comprising:
obtaining, with respect to each pair of one of the embedded areas and the corresponding one of the exposed areas, a sub-initial value and a sub-shrinkage value, and calculating a sub-assessment using the plurality of sub-initial values and the plurality of sub-shrinkage values for all of the pairs of the embedded areas and the exposed areas.

3. The method as claimed in claim 1, further comprising:
obtaining an auxiliary curve on the teeth section by detecting, for each of the exposed areas, at least one edge pixel at which a grayscale gradient value with an adjacent pixel is the largest among the pixels in the teeth section and which is located near the contour of the exposed area, resulting in a plurality of edge pixels, and then connecting the edge pixels to obtain the auxiliary curve.

4. The method as claimed in claim 3, further comprising, prior to step a), preparing a neural network using TensorFlow as a deep learning framework and compiled using Python.

5. The method as claimed in claim 4, wherein the preparing of the neural network 104A includes using a plurality of labeled images to train the neural network, each of the labeled images having a first curve which corresponds with the first reference curve of the to-be-processed image, a second curve which corresponds with the second reference curve of the to-be-processed image, an auxiliary curve which corresponds with the auxiliary curve of the to-be-processed image, and a third curve which corresponds with the alveolar boundary curve of the to-be-processed image.

6. The method as claimed in claim 1, further comprising, prior to step a), preparing a neural network using TensorFlow as a deep learning framework and compiled using Python.

7. The method as claimed in claim 6, wherein the preparing of the neural network includes using a plurality of labeled images to train the neural network, each of the labeled images having a first curve which corresponds with the first reference curve of the to-be-processed image, a second curve which corresponds with the second reference curve of the to-be-processed image, and a third curve which corresponds with the alveolar boundary curve of the to-be-processed image.
